(19) United States
(12) Patent Application Publication
(10) Pub. No.: US 2008/0124282 A1
(43) Pub. Date: May 29, 2008

(54) Oil-containing deodorizing aerosol compositions having skin-cooling active substances

(76) Inventors: Winfried EMMERLING, Tornesch (DE); Ulrike HEINSOHN, Hamburg (DE)

Correspondence Address: PAUL & PAUL, 2000 MARKET STREET, PHILADELPHIA, PA, 19103-3229, US

(21) Appl. No.: 11/948,192

(22) Filed: Nov. 30, 2007

Related U.S. Application Data
(63) Continuation of application No. PCT/EP2006/004931, filed on May 24, 2006.

(30) Foreign Application Priority Data
Jun. 1, 2005 (DE) 10 2005 025 495.0

(51) Int. Cl. A61K 8/06

(52) U.S. Cl. 424/47

(57) ABSTRACT

Essentially alcohol-free deodorizing aerosol compositions which contain selected oils, a skin-cooling active substance, a deodorizing active substance and a propellant gas.

### CROSS-REFERENCE TO RELATED APPLICATIONS

This application is a continuation under 35 U.S.C. § 365(c) and 35 U.S.C. § 120 of International Application No. PCT/EP2006/004931, filed May 24, 2006. This application also claims priority under 35 U.S.C. § 119 of German Patent Application No. DE 10 2005 025 495.0, filed Jun. 1, 2005. Both the International Application and the German Application are incorporated herein by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The subject matter of the present invention concerns essentially alcohol-free, oil-containing deodorizing aerosol compositions that comprise at least one skin-cooling active substance and exhibit a particularly high nurturing effect and compatibility to the skin.

In addition to antimicrobial and/or perspiration reducing active substances, deodorizing aerosol compositions generally comprise a specific type of perfume, whose function, in addition to making the product distinctive, is mainly the masking of odor. Nowadays, however, the consumer also expects additional benefits from a cosmetic product, in particular, good skin nurturing characteristics and a high compatibility to the skin, especially for sensitive skin. As the skin in the region of the underarm is often more sensitive than the facial skin, the problem of compatibility to the skin when developing underarm products is particularly important.

Moreover, one frequently wants cosmetic products, above all deodorants or antiperspirants, to have a freshening effect.

(2) Description of Related Art, Including Information Disclosed Under 37 C.F.R. §§ 1.97 and 1.98

With commercially available deodorant sprays, a large part of the freshening action is produced by means of a content of volatile solvents, especially ethanol or volatile silicone oils, such as cyclomethicones. A high ethanol content can indeed lead to hypersensitive reactions on the particularly sensitive skin of the underarm. A high content of cyclomethicones or other volatile silicone oils, especially short chain linear silicone oils such as hexamethyldisiloxane, octamethyltrisiloxane and decamethyltetrasiloxane for producing the desired cooling effect is disadvantageous because of cost. Antiperspirant sprays were known from U.S. Pat. No. 4,174,386, which, due to a high content of room temperature liquid pentane or other propellant gases generate a skin-cooling effect. However, these types of propellant gases lower the vapor pressure of the total propellant mixture and can result in an incomplete emptying of the aerosol container.

### BRIEF SUMMARY OF THE INVENTION

The object of the present invention is to provide deodorizing aerosol compositions that exhibit a refreshing skin sensation and at the same time a high compatibility with the skin. A further object of the present invention is to provide aerosol compositions with improved, especially longer lasting, deodorant power and/or freshening sensation.

Now, it was surprisingly found that by incorporating at least one skin-cooling active substance into a liquid carrier of selected oily substances and deodorant active substances and filling into an aerosol container with a propellant gas, particularly skin compatible and nurturing deodorizing aerosol compositions can be manufactured having a particular refreshing effect on the skin and an especially long lasting deodorant action. In this way the inventive deodorant sprays produce, even without ethanol, a high and at the same time pleasant refreshing effect and an excellent deodorant power.

### BRIEF DESCRIPTION OF THE SEVERAL VIEWS OF THE DRAWINGS

Not Applicable

### DETAILED DESCRIPTION OF THE INVENTION

Therefore, the subject matter of the present invention is a deodorizing aerosol composition that comprises at least one oil selected from the optionally hydroxylated esters of linear or branched saturated or unsaturated fatty alcohols containing 2-30 carbon atoms with linear or branched saturated or unsaturated, fatty acids containing 2-30 carbon atoms, from the benzoic acid esters of linear or branched C₈₋₂₂ alkanols, from the C₈-C₂₂ fatty alcohol esters of monovalent or polyvalent C₂-C₇ hydroxycarboxylic acids, branched saturated or unsaturated fatty alcohols containing 6-30 carbon atoms, dicarboxylic acid esters of linear or branched C₂-C₁₀ alkanols, di-n-alkyl ethers containing a total of 12 to 36 carbon atoms, at least one skin-cooling active substance, at least one deodorizing active substance and at least one propellant gas, wherein ethanol, isopropanol, 1-propanol and/or 2-propanol are comprised in total amounts of less than 0.5 wt. %.

The inventive compositions comprise at least one oil, selected from the following groups:
the optionally hydroxylated esters of linear or branched saturated or unsaturated fatty alcohols containing 2-30 carbon atoms with linear or branched saturated or unsaturated fatty acids containing 2-30 carbon atoms. These preferably include 2-ethylhexyl palmitate (e.g., Cegesoft® C 24), hexyldecyl stearate (Eutanol® G 16), hexyldecyl laurate, isodecyl neopentanoate, isononyl isononanoate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and -dipalmitate as well as mixtures thereof;
the benzoic acid esters of linear or branched C₈₋₂₂ alkanols, wherein C₁₂-C₁₅ alkyl benzoates, e.g., the commercial product Finsolv® TN, isostearyl benzoate, e.g., the commercial product Finsolv® SB, and ethylhexyl benzoate, e.g., the commercial product Finsolv® EB, as well as mixtures thereof are particularly preferred;
the C₈-C₂₂ fatty alcohol esters of monovalent or polyvalent C₂-C₇ hydroxycarboxylic acids, particularly preferably the esters of glycolic acid, lactic acid, malic acid, tartaric acid, citric acid and salicylic acid as well as mixtures thereof. Particularly preferred esters based on linear C_{12/15} alkanols, e.g., C₁₂-C₁₅ alkyl lactate, and C_{12/13} alkanols branched in the 2-position, e.g., di-C₁₂-C₁₃ alkyl malate, are obtained under the trade name Cosmacol® from Firma Nordmann, Rassmann GmbH & Co, Hamburg, particularly the commercial products Cosmacol® EMI, Cosmacol® ESI and Cosmacol® ETI;
the branched saturated or unsaturated fatty alcohols containing 6-30 carbon atoms. These alcohols are often called Guerbet alcohols as they are obtained by the Guerbet reaction. Particularly preferred alcoholic oils are, for example, hexyldecanol (e.g., Eutanol® G), octyldodecanol and 2-ethylhexyl alcohol as well as mixtures thereof;
dicarboxylic acid esters of linear or branched C₂-C₁₀ alkanols, particularly preferably diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate as well as mixtures thereof;
di-n-alkyl ethers containing in total 12 to 36, particularly 12 to 24 carbon atoms, wherein di-n-octyl ether (e.g., Cetiol® OE), di-n-decyl ether, n-hexyl n-octyl ether and n-octyl n-decyl ether as well as mixtures thereof are particularly preferred.

Particularly preferred inventive deodorizing aerosol compositions comprise an optionally hydroxylated ester of linear or branched saturated or unsaturated fatty alcohols containing 2-30 carbon atoms with linear or branched saturated or unsaturated fatty acids containing 2-30 carbon atoms, selected from 2-ethylhexyl palmitate, hexyldecyl stearate, hexyldecyl laurate, isodecyl neopentanoate, isononyl isononanoate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and -dipalmitate as well as mixtures thereof.

Further particularly preferred inventive deodorizing aerosol compositions comprise at least one benzoic acid ester of linear or branched C₈₋₂₂ alkanol, selected from C₁₂-C₁₅ alkyl benzoates, isostearyl benzoate and ethylhexyl benzoate as well as mixtures thereof.

Further particularly preferred inventive deodorizing aerosol compositions comprise at least the C₈-C₂₂ fatty alcohol esters of monovalent or polyvalent C₂-C₇ hydroxycarboxylic acids, selected from the C₈-C₂₂ fatty alcohol esters of glycolic acid, lactic acid, malic acid, tartaric acid, citric acid and salicylic acid as well as mixtures thereof.

Further particularly preferred inventive deodorizing aerosol compositions comprise at least one branched saturated or unsaturated fatty alcohol containing 6-30 carbon atoms, selected from hexyldecanol, octyldodecanol and 2-ethylhexyl alcohol as well as mixtures thereof.

Further particularly preferred inventive deodorizing aerosol compositions comprise at least one dicarboxylic acid ester of linear or branched C₂-C₁₀ alkanols, selected from diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate as well as mixtures thereof.

Further particularly preferred inventive deodorizing aerosol compositions comprise at least one di-n-alkyl ether containing a total of 12 to 36 carbon atoms, selected from di-n-octyl ether, di-n-decyl ether, n-hexyl n-octyl ether and n-octyl n-decyl ether as well as mixtures thereof.

Particularly preferred inventive deodorizing aerosol compositions comprise at least one of the cited oils in total amounts of 0.5-15 wt. %, preferably 1-10 wt. % and particularly preferably 3-7 wt. %, wherein amounts of 5-6 wt. % are exceedingly preferred.

Preferred inventive deodorizing aerosol compositions comprise at least one skin-cooling active substance, selected from menthol, isopulegol as well as menthol derivatives, preferably menthyl lactate, menthol propylene glycol carbonate, menthol ethylene glycol carbonate, menthyl pyrrolidone carboxylic acid, menthyl methyl ether, menthoxypropane diol, menthone glycerine acetal (9-methyl-6-(1-methylethyl)-1,4-dioxaspiro-(4.5)decan-2-methanol), monomenthyl succinate, menthyl glycolate and 2-hydroxymethyl-3,5,5-trimethylcyclohexanol as well as mixtures thereof. Preferred skin-cooling active substances are menthol, isopulegol, menthyl lactate, menthol propylene glycol carbonate, menthol ethylene glycol carbonate, menthone glycerine acetal (e.g., obtainable from Symrise under the trade name Frescolat MGA), menthoxypropane diol and menthyl pyrrolidone carboxylic acid as well as mixtures thereof.

An inventively exceedingly preferred mixture of skin-cooling active substances is selected from menthol propylene glycol carbonate, menthol ethylene glycol carbonate and menthol, such as, for example, obtained under the trade name Optacool A from the Symrise Company.

Particularly preferred inventive deodorizing aerosol compositions comprise at least one skin-cooling active substance in total amounts of 0.01-1 wt. %, preferably 0.02-0.5 wt. % and particularly preferably 0.05-0.2 wt. %, each based on the total weight of the total weight of the aerosol composition.

Particularly preferred inventive deodorizing aerosol compositions comprise at least one encapsulated skin-cooling active substance.

Preferred inventive deodorizing aerosol compositions comprise at least one deodorizing active substance, selected from odor absorbers, deodorizing ion exchangers, germ inhibitors, prebiotic active components as well as inhibitors of the enzymes that are responsible for the decomposition of perspiration or, particularly preferably, combinations of these active substances.

Silicates serve as odor absorbers and at the same time also advantageously support the rheological properties of the inventive composition. The particularly preferred silicates according to the invention particularly include layered silicates and among these particularly montmorillonite, kaolinite, illite, beidellite, nontronite, saponite, bentonite, smectite and talcum. Further particularly preferred odor absorbers are, for example, zeolites, zinc ricinoleate, cyclodextrins, certain metal oxides, such as, e.g., aluminum oxide, as well as chlorophyll. They are preferably added in an amount of 0.1-10 wt. %, particularly preferably 0.5-7 wt. % and exceedingly preferably 1-5 wt. %, each based on the total composition.

In accordance with the invention, germ inhibitors or antimicrobials are understood to mean those active substances that reduce the number of the skin germs involved in odor formation or that inhibit their growth. These germs include inter alia different species from the group of the staphylococci (e.g., Staphylococcus hominis), from the group of the corynebacteria (e.g., Corynebacterium xerosis, Corynebacterium CDCG2), anaerococci (e.g., Anaerococcus octavius) and micrococci.

According to the invention, especially the mixtures of odoriferous substances Protectate HR and Protectate MOD 2 from the Symrise Company are particularly preferred germ inhibitors or antimicrobials. The inventively preferred mixture of odoriferous substances Protectate HR from the Symrise Company comprises 25-50 wt. % phenoxyethanol, 5-10 wt. % 2-methyl-5-phenylpentan-1-ol with the trivial name Rosaphen, 34-70 wt. % 2-benzylheptan-1-ol with the trivial name Jasmol, 1-5 wt. % 4-methoxybenzyl alcohol (anise alcohol) and 0.01-1 wt. % 5-methyl-2-isopropylphenol (thymol). The inventively particularly preferred mixture of odoriferous substances Protectate MOD 2 from the Symrise Company comprises 25-45 wt. % phenoxyethanol, 5-10 wt. % 2-methyl-5-phenylpentan-1-ol and 45-70 wt. % 2-benzylheptan-1-ol.

Particularly preferred inventive deodorizing aerosol compositions comprise at least one of the mixtures of odoriferous substances Protectate HR or Protectate MOD 2 in total amounts of 0.001 to 5 wt. %, preferably 0.05 to 2 wt. % and particularly preferably 0.1 to 1.0 wt. %, each based on the total weight of the cosmetic composition.

Moreover, according to the invention, preferred germ-inhibitors or antimicrobials are organo halogen compounds as well as organo halides, quaternary ammonium compounds, a series of plant extracts and zinc compounds. They include, inter alia, triclosan, chlorhexidine and chlorhexidine gluconate, 3,4,4′-trichlorocarbanilide, bromochlorophene, dichlorophene, chlorothymol, chloroxylenol, hexachlorophene, dichloro-m-xylene, dequalinium chloride, domiphen bromide, ammonium phenolsulfonate, benzalkonium halides, benzalkonium cetyl phosphate, benzalkonium saccharinate, benzethonium chloride, cetylpyridinium chloride, laurylpyridinium chloride, laurylisoquinolinium bromide, methylbenzedonium chloride. In addition, preferred deodorizing active substances are phenol, phenoxyethanol, disodium dihydroxyethyl sulfosuccinyl undecylenate, sodium bicarbonate, zinc lactate, sodium phenolsulfonate and zinc phenolsulfonate, keto glutaric acid, terpene alcohols such as, e.g., the particularly preferred farnesol, chlorophyl-copper complexes, α-monoalkyl glycerine ethers with a branched or linear saturated or unsaturated, optionally hydroxylated C₆-C₂₂ alkyl group, particularly preferably α-(2-ethylhexyl) glycerine ether, commercially available as Sensiva® SC 50 (from Schulke & Mayr), carboxylic acid esters of mono-, di- and triglycerine (e.g., glycerine monolaurate, diglycerine monocaprinate), lantibiotics as well as plant extracts (e.g., green tea and constituents of lime tree blossom oil).

Further preferred deodorizing active substances are selected from the “prebiotically” active components, under which, according to the invention, are understood to be those components that inhibit only, or at least predominantly, the odor-forming germs of the skin microflora, but not the desired, i.e., the non-odor forming germs that belong to a healthy skin microflora. The active substances disclosed in the Offenlegungsschriften DE 10333245 and DE 10 2004 011 968 as prebiotically active are explicitly included herein; they include conifer extracts, especially from the group of the Pinaceae, and plant extracts from the group of the Sapindaceae, Araliaceae, Lamiaceae and Saxifragaceae, especially extracts from Picea spp., Paullinia sp., Panax sp., Lamium album or Ribes nigrum as well as mixtures of these substances.

Further preferred deodorizing active substances are selected from the germ-inhibiting perfume oils and the deosafe perfume oils, which can be obtained from the Symrise Company, previously known as Haarmann and Reimer.

Deodorizing enzyme inhibitors are those substances that inhibit the enzymes responsible for decomposing the perspiration, especially arylsulfatase, β-glucuronidase, amino acylase, ester cleaving lipases and lipoxigenase, wherein trialkyl citric acid esters, especially triethyl citrate, or zinc glycinate, are exceedingly preferred.

Exceedingly preferred deodorizing active substances are selected from the mixtures of odoriferous substances Protectate HR and Protectate MOD 2 from the Symrise Company, phenoxyethanol, farnesol, α-(2-ethylhexyl) glycerine ether, glycerine monolaurate, diglycerine monocaprinate, triethyl citrate, conifer extracts from the group of the Pinaceae, plant extracts from the group of the Sapindaceae, Araliaceae, Lamiaceae and Saxifragaceae, the deosafe perfume oils from the Symrise Company as well as mixtures of these substances.

It was surprisingly found that these deodorizing active agents in combination with skin-cooling active substances, especially mixtures of the three components menthol propylene glycol carbonate, menthol ethylene glycol carbonate and menthol, and the inventively used oils show a particularly long lasting deodorant power and/or a particularly long lasting refreshing feeling.

Particularly preferred inventive deodorizing aerosol compositions are characterized in that they comprise at least one deodorizing active substance in a total amount of 0.1-10 wt. %, preferably 0.2-7 wt. %, particularly preferably 0.3-5 wt. % and exceedingly preferably 0.4-1.0 wt. %, based on the total weight of the total weight of the composition.

Particularly preferred inventive deodorizing aerosol compositions are characterized in that they comprise at least one encapsulated and/or at least one non-encapsulated fragrance. The encapsulation of the skin-cooling active substance and/or the fragrance can preferably be chosen such that it involves a water-soluble encapsulating material. A certain time after the application the effect of moisture, in this case especially the effect of skin moisture or perspiration, causes the water-soluble encapsulating material to open up, thereby releasing the encapsulated fragrance as well as optionally additional active substances, e.g., skin-cooling active substances, with a delay after the application.

Encapsulated and non-encapsulated fragrances, for example perfume oils or mixtures of perfume oils, may be the same or different. Particularly preferred inventive deodorizing aerosol compositions comprise at least one encapsulated and at least one non-encapsulated fragrance, which differ from one another.

Particularly preferred inventive deodorizing aerosol compositions comprise at least one non-encapsulated fragrance in a total amount of 0.1-3 wt. %, preferably 0.2-1.5 wt. % and particularly preferably 0.4-1 wt. %, each based on the total weight of the aerosol composition. Further particularly preferred inventive deodorizing aerosol compositions comprise at least one encapsulated fragrance in a total amount of 0.01-2 wt. %, preferably 0.1-1.0 wt. % and particularly preferably 0.25-0.5 wt. %, each based on the total weight of the aerosol composition.

In a further particularly preferred embodiment of the invention, the encapsulated fragrance and the encapsulated skin-cooling active substance are present as a combined encapsulated mixture. However, both components may also be separately encapsulated side by side. In a preferred embodiment of the invention, fragrance and skin-cooling active substance are encapsulated together as a mixture.

According to the invention, the weight ratio of encapsulated fragrance and encapsulated skin-cooling active substance preferably ranges from 10:1 to 1:10.

Particularly preferred fragrances or perfume oils that are used are odoriferous compounds, for example, synthetic products of the ester, ether, aldehyde, ketone, alcohol and hydrocarbon type. The preferred phenolic odoriferous compounds include, e.g., carvacrol. Preferred odoriferous compounds of the ester type are, for example, benzyl acetate, methyl anthranilate, ortho-t-butylcyclohexyl acetate, p-tert.-butylcyclohexyl acetate, diethyl phthalate, nonane-1,3 diol diacetate, iso-nonyl acetate, iso-nonyl formate, phenylethylphenyl acetate, phenoxyethyl isobutyrate, linalyl acetate, dimethylbenzyl carbinyl acetate, phenylethyl acetate, linalyl benzoate, benzyl formate, ethylmethylphenyl glycinate, allylcyclohexyl propionate, styrallyl propionate, benzyl salicylate, ethyl salicylate, iso-amyl salicylate and 4-nonanolide. The preferred ethers include, for example, benzyl ethyl ether; the preferred aldehydes include, for example, the linear alkanals containing 8 to 18 carbon atoms, citral, citronellal, citronellyloxyacetaldehyde, cyclamen aldehyde, hydroxycitronellal, lilial and bourgeonal; the preferred ketones include, for example, 6-acetyl-1,1,3,4,4,6-hexamethyltetrahydronaphthalene, para-t-amylcyclohexanone, 2-n-heptylcyclopentanone, β-methylnaphthyl ketone and the ionones α-isomethylionone and methyl cedryl ketone; the preferred alcohols include cinnamyl alcohol, anethol, citronellol, dimyrcetol, eugenol, geraniol, linalool, phenylethyl alcohol and terpineol; the preferred hydrocarbons include 1,3,4,6,7,8-hexahydro-4,6,6,7,8,8-hexamethylcyclopenta-α-2-benzopyran, hydroxymethylisopropylcyclopentane, 3-α-methyldodecahydro-6,6,9a-trimethylnaphtho-2(2,1-b)furan, iso-butylquinoline as well as the terpenes and balsams. Mixtures of various odoriferous substances, which together produce an attractive fragrant note, are particularly preferred.

Particularly preferred perfume oils can also comprise natural mixtures of odoriferous substances as are obtainable from vegetal or animal sources, for example, pine oil, citrus oil, jasmine oil, ylang-ylang oil, rose oil or oil of lillies. The essential oils of lower volatility that are mostly used as aroma components are particularly preferred as the perfume oils, e.g., oil of sage, chamomile oil, clove oil, melissa oil, mint oil, cinnamon leaf oil, lime blossom oil, juniper berry oil, vetivert oil, olibanum oil, galbanum oil, laudanum oil, oil of clove buds, iso-eugenol, oil of thyme, rose oil, bergamot oil and geranium oil.

The capsule materials are preferably water-soluble polymers such as starch, physically and/or chemically modified starches, cellulose derivatives such as, e.g., carboxymethyl cellulose, methyl cellulose, hydroxyethyl cellulose or hydroxypropyl methyl cellulose, carragheen, alginates, maltodextrines, dextrines, plant gums, pectines, xanthanes, polyvinyl acetate and polyvinyl alcohol, polyvinyl pyrrolidine, polyamides, polyesters and homopolymers and copolymers of monomers, selected from acrylic acid, methacrylic acid, maleic acid, fumaric acid, itaconic acid as well as the esters and the salts of these acids, as well as any mixtures of these polymers.

Particularly preferred capsule materials are chemically modified starches, especially aluminum starch octenylsuccinate, e.g., the commercial product Dry Flo Plus from National Starch, or sodium starch octenylsuccinate, e.g., the commercial product Tylose H 10 from Clariant, furthermore the carboxymethyl celluloses, carboxymethyl cellulose, methyl cellulose, hydroxyethyl cellulose and hydroxypropyl methyl cellulose, in addition carragheen, alginates and maltodextrines, as well as any mixtures of these polymers.

Particularly preferred capsule materials are polymer mixtures that consist of chemically modified starches and/or hydroxyethyl cellulose and a 0.2-2 wt. % content of alginates and/or carragheen.

The encapsulation can be carried out by known processes. Suitable processes are disclosed in e.g., K. Master, “Spray Drying Handbook,” 3rd Edition, John Wiley, 1979. In a particularly preferred encapsulation process, a water-based mixture is manufactured that comprises about 20-50 wt. % of the polymeric encapsulating material, about 0.1-2.0 wt. % of an emulsifier, about 5-20 wt. % of the perfume oil and/or the skin-cooling active substance to be encapsulated as well as 40-60 wt. % water. This mixture is homogenized and subsequently spray dried. The capsules loaded with active substance are thus obtained as a fine powder with a particle diameter of 1-150 μm, preferably 20-80 μm, particularly preferably 5-50 μm.

In another manufacturing process, the micro encapsulation results from coacervation, wherein the carrier is preferably formed from gelatine.

The capsule material, consisting of water-soluble polymers and a low content of emulsifiers, allows a reversible “re-encapsulation” of the encapsulated perfume oil and skin-cooling active substances. The re-encapsulation occurs in situ as the skin dries, following a period of perspiration. Thus, different consecutive activations happen on the skin without the need for the consumer applying an additional application of the inventive composition.

According to the invention, fragrance-free or perfume-free deodorizing aerosol compositions can be preferred.

The inventive compositions comprise ethanol, isopropanol and/or propanol in total amounts of less than 0.5 wt. %, based on the total aerosol composition. Preferred inventive deodorizing aerosol compositions comprise less than 0.2 wt. %, particularly preferably 0 wt. % ethanol, isopropanol, 1-propanol and/or 2-propanol, each based on the total aerosol composition.

Particularly preferred inventive deodorizing aerosol compositions are essentially anhydrous, i.e., they comprise 0 wt. % or maximum 2 wt. % water, based on the total aerosol composition.

Further particularly preferred inventive deodorizing aerosol compositions are present as a water-in-oil emulsion or as an oil-in-water emulsion. The water content is then 5-90 wt. %, preferably 10-80 wt. %, particularly preferably 30-60 wt. %, in each case based on the weight of the propellant gas-free emulsion.

Preferred inventive deodorizing aerosol compositions are those in which the at least one propellant gas is selected from n-propane, n-butane, isobutane, n-pentane, isopentane, dimethyl ether, carbon dioxide, nitrous oxide, fluorinated hydrocarbons and fluorochlorinated hydrocarbons as well as mixtures of these substances.

Particularly preferred inventive deodorizing aerosol compositions comprise the at least one propellant gas in total amounts of 20-95 wt. %, preferably 30-95 wt. % and exceedingly preferably 60-95 wt. %, each based on the total aerosol composition.

Particularly preferred inventive deodorizing aerosol compositions are those wherein the weight ratio of oil and skin-cooling, deodorizing and optionally additional active substances that are dissolved therein to the propellant gas in the inventive compositions preferably ranges from 50: 50-5:95, particularly preferably 30: 70-10:90.

Particularly preferred inventive deodorizing aerosol compositions comprise at least one antiperspirant active substance. Inventively preferred antiperspirant active substances are the water-soluble astringent inorganic and organic salts of aluminum, zirconium and zinc or any mixtures of these salts. Particularly preferred antiperspirant active substances are selected from aluminum chlorohydrates, for example, aluminum sesquichlorohydrate, aluminum chlorohydrex-propylene glycol (pg) or -polyethylene glycol (peg), aluminum sesquichlorohydrex-pg or -peg, aluminum pg-dichlorohydrex or aluminum peg-dichlorohydrex, aluminum hydroxide, additionally selected from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum-zirconium chlorohydrate-glycine complexes such as aluminum zirconium trichlorohydrexglycine, aluminum zirconium tetrachlorohydrexglycine, aluminum zirconium pentachlorohydrexglycine, aluminum zirconium octachlorohydrexglycine, potassium aluminum sulfate (KAl(SO₄)₂12H₂O, alum), aluminum undecylenoyl collagen amino acid, sodium aluminum lactate+aluminum sulfate, sodium aluminum chloro hydroxylactate, aluminum bromohydrate, aluminum chloride, the complexes of zinc and sodium salts, the complexes of lanthanum and cerium, the aluminum salts of lipoamino acids, aluminum sulfate, aluminum lactate, aluminum chlorohydroxy allantoinate, sodium aluminum chlorohydroxy lactate, zinc chloride, zinc sulfocarbolate, zinc sulfate and zirconium chlorohydrate. According to the invention, water-solubility is understood to mean a solubility of at least 5 wt. % at 20° C., i.e., that quantities of at least 5 g of the antiperspirant active substance are soluble in 95 g water at 20° C. The antiperspirant active substances can be used as aqueous solutions. Particularly preferred inventive deodorizing aerosol compositions comprise at least one antiperspirant active substance in a total amount of 3-25 wt. %, preferably 5-22 wt. % and particularly 10-20 wt. %, each based on the weight of the active substance in the total composition. In a preferred embodiment, the composition comprises an astringent aluminum salt, especially aluminum chlorohydrate, which is commercially available, for example, in powder form as Micro Dry® Ultrafine from Reheis, as Chlorhydrol® as well as in activated form as Reach® 501 from Reheis.

Further particularly preferred inventive deodorizing aerosol compositions comprise at least one suspending agent or thickener, selected from hydrophobized clay minerals and pyrogenic silicic acids. Preferred hydrophobized clay minerals are montmorillonites, hectorites and bentonites, especially disteardimonium hectorite and quaternium-18 hectorite. The commercial thickeners make available these hydrophobized clay minerals in the form of a gel in cyclomethicone and optionally with an additional oil component, such as propylene carbonate. Further preferred thickeners are pyrogenic silicic acids, e.g., the commercial products of the Aerosil® series from Degussa.

The inventive compositions are preferably packaged in ordinary aerosol cans. The cans can be made from tin plate or from aluminum. Moreover, according to a particularly preferred embodiment, the cans can be internally coated in order to control the danger of corrosion as much as possible.

Preferably the aerosol cans are equipped with a suitable spraying head. Depending on the spraying head, the discharge rates, based on a fully filled can, are preferably from 0.1 g/s to 2.0 g/s.

The following examples are intended to clarify the invention without limiting it in any way.

Inventive exemplary compositions (all quantities are in wt. %).

| COMPOSITION | 1 | 2 | 3 | 4 | 5 | 6 | 7 | 8 | 9 | 10 |
|---|---|---|---|---|---|---|---|---|---|---|
| Isopropyl myristate | 5.0 | — | — | — | — | — | 3.0 | — | — | — |
| Ethylhexyl palmitate | — | 5.0 | — | — | — | — | — | 2.5 | — | — |
| Cetiol ® OE | — | — | 4.0 | — | — | — | — | — | — | — |
| Cosmacol EMI | — | — | — | 3.8 | — | — | 2.0 | — | 5.0 | — |
| Finsolv TN | — | — | 1.0 | — | — | — | — | — | — | 4.5 |
| n-Hexyl laurate | — | — | — | — | 3.0 | — | — | 1.0 | — | — |
| Isopropyl palmitate | — | — | — | — | 2.0 | — | — | — | — | — |
| Isopropyl isostearate | — | — | — | — | — | 4.5 | — | — | — | — |
| Triethyl citrate | 2.9 | 1.0 | 0.5 | — | — | — | 3.5 | 2.1 | 1.2 | — |
| Phenoxyethanol | 0.5 | — | — | — | 1.0 | — | — | — | — | — |
| Protectate HR | 0.2 | — | — | — | — | 0.3 | 0.5 | — | — | — |
| Protectate MOD 2 | — | — | 0.5 | — | 0.5 | — | — | — | — | 0.7 |
| α-(2-Ethylhexyl) glycerine ether | 0.3 | — | — | 1.0 | 0.4 | — | — | — | 1.0 | — |
| Glycerine monolaurate | — | 0.5 | 0.3 | — | — | 0.5 | — | — | — | — |
| Diglycerine monocaprinate | 0.2 | — | — | 1.0 | — | — | 0.3 | 0.5 | — | 0.3 |
| Farnesol | — | — | — | 0.5 | — | — | 0.3 | 0.5 | 0.2 | 0.2 |
| Ginseng extract in propylene glycol | — | — | — | — | 0.4 | — | — | 0.2 | — | — |
| Deosafe perfume oil | — | — | — | — | 0.5 | — | — | 0.5 | — | 1.0 |
| Optacool A (non-encapsulated) | 0.1 | 0.1 | 0.1 | 0.2 | — | — | — | — | — | — |
| Isopulegol | — | — | — | — | 0.3 | — | — | — | 0.2 | — |
| Menthyl lactate (encapsulated) | — | — | — | — | 0.3 | 0.5 | — | — | — | 0.3 |
| Menthyl methyl ether | — | — | — | — | — | — | 0.5 | — | — | — |
| Menthyl pyrrolidone carboxylic acid | — | — | — | — | — | 0.1 | — | 0.5 | — | — |
| Perfume (non-encapsulated) | 1.0 | 0.5 | 2.0 | — | — | 1.0 | 1.0 | — | 2.0 | — |
| Perfume (encapsulated) | — | 0.5 | — | — | 1.0 | — | 1.0 | 1.0 | — | — |
| REACH 103 | — | — | — | — | — | — | — | — | — | 5 |
| (activated aluminum chlorohydrate, Reheis) | | | | | | | | | | |
| n-Pentane | — | 5 | — | — | — | — | — | — | 5 | — |
| Isopentane | — | 5 | — | — | — | — | — | — | — | 5 |
| Isobutane | — | — | — | — | 30 | — | — | — | — | — |
| n-Propane | — | — | — | 20 | — | — | 20 | — | — | — |
| n-Butane | ad | ad | ad | ad | ad | ad | ad | ad | ad | ad |
| | 100 | 100 | 100 | 100 | 100 | 100 | 100 | 100 | 100 | 100 |

Compositions 1-5 were filled into internally coated tin plate cans; compositions 6-10, in internally coated aluminum cans.

1. A deodorizing aerosol composition, comprising
a) at least one oil, selected from the group consisting of optionally hydroxylated esters of linear or branched saturated or unsaturated fatty alcohols containing 2-30 carbon atoms with linear or branched saturated or unsaturated fatty acids containing 2-30 carbon atoms, benzoic acid esters of linear or branched C₈₋₂₂ alkanols, C₈-C₂₂ fatty alcohol esters of monovalent or polyvalent C₂-C₇ hydroxycarboxylic acids, branched saturated or unsaturated fatty alcohols containing 6-30 carbon atoms, dicarboxylic acid esters of linear or branched C₂-C₁₀ alkanols, di-n-alkyl ethers containing in total 12 to 36 carbon atoms,
b) at least one skin-cooling active substance,
c) at least one deodorizing active substance, and
d) at least one propellant gas,
wherein ethanol, isopropanol, 1-propanol and/or 2-propanol are comprised in total amounts of less than 0.5 wt. %.

2. The composition according to claim 1, wherein the at least one optionally hydroxylated ester of linear or branched saturated or unsaturated fatty alcohols containing 2-30 carbon atoms with linear or branched saturated or unsaturated fatty acids containing 2-30 carbon atoms, is selected from the group consisting of 2-ethylhexyl palmitate, hexyldecyl stearate, hexyldecyl laurate, isodecyl neopentanoate, isononyl isononanoate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and -dipalmitate as well as mixtures thereof.

3. The composition according to claim 1, wherein the at least one benzoic acid ester of linear or branched C₈₋₂₂ alkanols is selected from the group consisting of C₁₂-C₁₅ alkyl benzoates, isostearyl benzoate and ethylhexyl benzoate as well as mixtures thereof.

4. The composition according to claim 1, wherein the at least one C₈-C₂₂ fatty alcohol ester of monovalent or polyvalent C₂-C₇ hydroxycarboxylic acids is selected from the group consisting of C₈-C₂₂ fatty alcohol esters of glycolic acid, lactic acid, malic acid, tartaric acid, citric acid and salicylic acid as well as mixtures thereof.

5. The composition according to claim 1, wherein the at least one branched saturated or unsaturated fatty alcohol containing 6-30 carbon atoms is selected from the group consisting of hexyldecanol, octyldodecanol and 2-ethylhexyl alcohol as well as mixtures thereof.

6. The composition according to claim 1, wherein the at least one dicarboxylic acid ester of linear or branched C₂-C₁₀ alkanols is selected from the group consisting of diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate as well as mixtures thereof.

7. The composition according to claim 1, wherein the at least one di-n-alkyl ether containing in total 12 to 36 carbon atoms is selected from the group consisting of di-n-octyl ether, di-n-decyl ether, n-hexyl n-octyl ether and n-octyl n-decyl ether as well as mixtures thereof.

8. The composition according to claim 1, wherein the at least one skin-cooling active substance is selected from the group consisting of menthol, isopulegol, menthol derivatives, menthyl lactate, menthol propylene glycol carbonate, menthol ethylene glycol carbonate, menthyl pyrrolidone carboxylic acid, menthyl methyl ether, menthoxypropane diol, menthone glycerine acetal (9-methyl-6-(1-methylethyl)-1,4-dioxaspiro-(4.5)decane-2-methanol), monomenthyl succinate, menthyl glycolate and 2-hydroxymethyl-3,5,5-trimethylcyclohexanol as well as mixtures thereof.

9. The composition according to claim 1, wherein the at least one deodorizing active substance is selected from the group consisting of odor absorbers, deodorizing ion exchangers, germ inhibitors, prebiotic active components, inhibitors of the enzymes that are responsible for the decomposition of perspiration and combinations of these active substances.

10. The composition according to claim 1 comprising at least one encapsulated skin-cooling active substance.

11. The composition according to claim 1 comprising at least one encapsulated fragrance and/or one non-encapsulated fragrance.

12. The composition according to claim 11 comprising at least one encapsulated and at least one non-encapsulated fragrance, which differ from one another.

13. The composition according to claim 1 comprising 0 wt. % ethanol, isopropanol, 1-propanol and/or 2-propanol, based on the total aerosol composition.

14. The composition according to claim 1 comprising a maximum of 2 wt. % water, based on the total aerosol composition.

15. The composition according to claim 1 additionally comprising at least one antiperspirant active substance.

16. The composition according to claim 1 additionally comprising at least one thickener, selected from hydrophobized clay minerals and pyrogenic silicic acids.

17. The composition according to claim 1 wherein said composition is present as a water-in-oil or as an oil-in-water emulsion.

18. An aerosol container comprising a deodorizing aerosol composition, comprising
a) at least one oil, selected from the group consisting of optionally hydroxylated esters of linear or branched saturated or unsaturated fatty alcohols containing 2-30 carbon atoms with linear or branched saturated or unsaturated fatty acids containing 2-30 carbon atoms, benzoic acid esters of linear or branched C₈₋₂₂ alkanols, C₈-C₂₂ fatty alcohol esters of monovalent or polyvalent C₂-C₇ hydroxycarboxylic acids, branched saturated or unsaturated fatty alcohols containing 6-30 carbon atoms, dicarboxylic acid esters of linear or branched C₂-C₁₀ alkanols, di-n-alkyl ethers containing in total 12 to 36 carbon atoms,
b) at least one skin-cooling active substance,
c) at least one deodorizing active substance, and
d) at least one propellant gas,
wherein ethanol, isopropanol, 1-propanol and/or 2-propanol are comprised in total amounts of less than 0.5 wt. %.

19. The aerosol container according to claim 18, wherein the at least one deodorizing active substance is selected from the group consisting of odor absorbers, deodorizing ion exchangers, germ inhibitors, prebiotic active components, inhibitors of the enzymes that are responsible for the decomposition of perspiration and combinations of these active substances.

20. A method of deodorizing and cooling of skin comprising the step of applying to the skin a deodorizing aerosol composition comprising
a) at least one oil, selected from the group consisting of optionally hydroxylated esters of linear or branched saturated or unsaturated fatty alcohols containing 2-30 carbon atoms with linear or branched saturated or unsaturated fatty acids containing 2-30 carbon atoms, benzoic acid esters of linear or branched C₈₋₂₂ alkanols, C₈-C₂₂ fatty alcohol esters of monovalent or polyvalent C₂-C₇ hydroxycarboxylic acids, branched saturated or unsaturated fatty alcohols containing 6-30 carbon atoms, dicarboxylic acid esters of linear or branched C₂-C₁₀ alkanols, di-n-alkyl ethers containing in total 12 to 36 carbon atoms,
b) at least one skin-cooling active substance,
c) at least one deodorizing active substance, and
d) at least one propellant gas
wherein ethanol, isopropanol, 1-propanol and/or 2-propanol are comprised in total amounts of less than 0.5 wt. %.
